(19) United States
(12) Patent Application Publication
(10) Pub. No.: US 2011/0152779 A1
(43) Pub. Date: Jun. 23, 2011

(54) Catheter Clip

(75) Inventor: Christos Panotopoulos, Athens (GR)

(73) Assignee: JAYMORE ENTERPRISES LIMITED, Limassol (CY)

(21) Appl. No.: 12/675,014

(22) PCT Filed: Aug. 1, 2008
(86) PCT No.: PCT/IB08/02009

§ 371 Date: Feb. 24, 2010

Related U.S. Application Data
(60) Provisional application No. 60/966,428, filed on Aug. 27, 2007.

(51) Int. Cl. A61M 25/02; A61B 19/02

(52) U.S. Cl. 604/180; 206/364

(57) ABSTRACT

The invention provides a device for securing an invasive medical implementation, such as a catheter, to a patient. The device generally combines an adherent surface that attaches the device to the skin and a flexible clasping means to grasp the catheter and reversibly secure it to the device.

### FIELD OF THE INVENTION

The invention generally relates to a device for securing an invasive medical implementation to a subject. Specific embodiments allow a user to secure a catheter to a patient rapidly and without the aid of prior surgical tapes, glues, dressings and/or foams.

### BACKGROUND

Medical implementations such as catheters, medical lines, tubing and like articles are routinely used to move fluids to and from patients. A catheter typically includes a hard part which remains exterior to the patient, and a soft part, at least a portion of which is inserted into the patient. The hard part can include a connection point (sometimes called a hub) to which other medical implementations (eg., a syringe, fluid supply tube) can be joined. Following installation and securement (“catheterization”), the catheter is a convenient means for administering fluids such as drugs or withdrawing blood or other body fluids from the patient.

According to prior practice, a caregiver uses glue, foam, surgical tape, dressing or a combination thereof to secure the catheter against the skin of the patient. Typically, the caregiver covers the catheter insertion site with a dressing after swabbing the area with antiseptic. The entire procedure can take several minutes or more. In addition, catheterization can require frequent disconnection between of the catheter as new medical lines are added or replaced, thereby stressing securing tapes, glues or foams. In settings involving long-term catheter use, the caregiver must frequently clean the insertion site about the inserted (indwelling) catheter, change the dressings, and apply fresh antiseptic.

There has been increasing recognition that caregivers spend too much time securing catheters to patients. Moreover, prior practice has not been able to prevent catheter dislodgement and/or infection near insertion sites. Catheters that are taped or glued in place are readily pulled out during “routine” dressing changes. Surgical tapes and foams can be uncomfortable or irritating for some. Many patients cannot rest comfortably knowing that a secured catheter may dislodge from the insertion time during sleep. Young or elderly patients are especially vulnerable to these and related shortcomings.

It is becoming clear that prior catheter securement devices and procedures have not optimally served patients. These and other drawbacks have created a need for a more rapid and reliable device for securing catheters to patients. Accordingly, it would be desirable to have a device that can be used to secure a catheter to the skin of a patient that does not rely on use of prior tapes, glues, dressings and/or foams. It would be further desirable to have methods of using such a device so that the catheter can be rapidly and reliably secured to the patient.

### SUMMARY

In broad terms, the invention provides a device for securing an invasive medical implementation such as a catheter to a patient. The device generally combines (1) an adherent surface that attaches the device to the skin and (2) a flexible clasping means to grasp the catheter and secure it to the device. Securement is typically reversible. Preferred practice of the invention avoids use of prior tapes, glues, dressings and/or foams to secure the medical implementation to the patient. Use of the invention can substantially reduce catheter securement times, thereby enhancing the reliability, comfort, and safety of catheterization. The invention is relatively simple to use and can be employed by experienced and inexperienced caregivers alike.

Accordingly, and in one aspect, the invention provides a device for securing a catheter to an insertion site of a patient. In one embodiment, the device includes at least one of and preferably all of the following as operably linked components:

(a) a flexible base comprising a sealed patient contacting surface,
(b) a solid portion joined to the flexible base and comprising a clasping means for securing the catheter; and
(c) a handle adapted to remove, with one digit (finger or thumb), a seal from the patient contacting surface. Preferably, the handle is joined to the seal which seal protects the adhesive on at least part of the surface sufficient to attach the secured catheter to the patient insertion site. In a particular invention embodiment, the device will be referred to herein as a “catheter clip” or like phrase.

In another aspect, the invention provides a unitary package that includes the device wherein the device is preferably sterile and includes a packing material substantially resistant to penetration by microorganisms, viruses and the like.

In yet another aspect, the invention provides a method of securing a catheter to an insertion site of a patient. In one embodiment, the method includes at least one of and preferably all of the following steps:

(a) providing a device comprising:

(i) a flexible base comprising a sealed patient contacting surface,

(ii) a solid portion joined to the flexible base and comprising a clasping means for securing the catheter to the device,

(iii) a handle region joined to the flexible portion and adapted to remove, with one hand, a seal from the patient contacting surface, thereby exposing the adhesive for releasably securing the catheter to the insertion site of the patient,

(b) removing, with at least one digit, the seal from the patient contacting surface of the flexible portion to expose the adhesive,
(c) guiding the device over the catheter,
(d) contacting the flexible base (patient contacting surface) to the patient sufficient to adhere the device thereto; and
(e) actuating the clasping means to grasp the catheter (preferably at or around the solid part), thereby securing the catheter to insertion site of the patient.

Further uses and advantages of the invention will be apparent from the following Drawings and discussion.

### BRIEF DESCRIPTION OF THE DRAWINGS

FIG. 1A is a drawing (top view) showing a catheter clip of the invention.

FIG. 1B is a drawing (top view) showing the catheter clip securing a catheter having lateral wings for stabilizing the catheters and connecting hubs.

FIG. 2 is a drawing (cross-sectional view) of the catheter clip securing a catheter against the skin of a patient.

### DETAILED DESCRIPTION

In the present section, the invention is illustrated with regard to one or more particular embodiments. These embodiments are intended merely to illustrate certain principles of the invention and not to limit the invention or use thereof in any way.

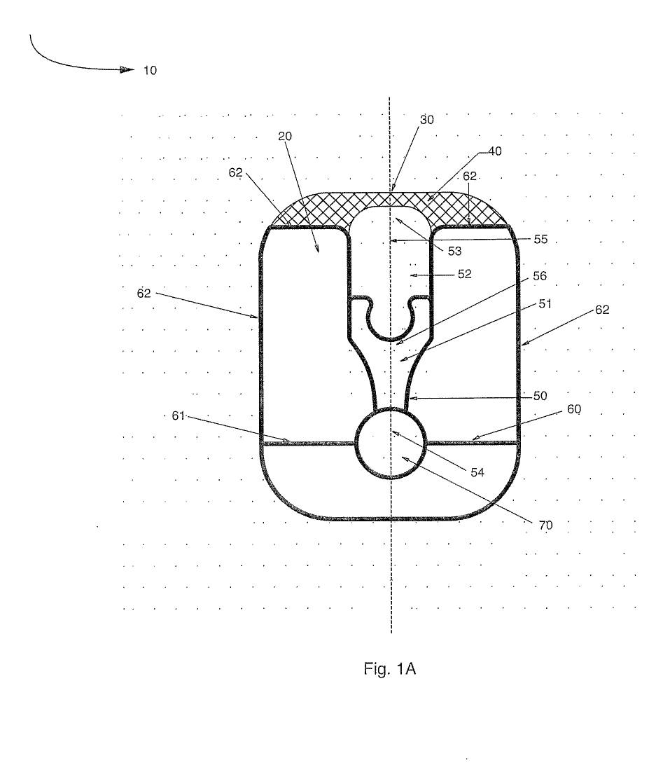

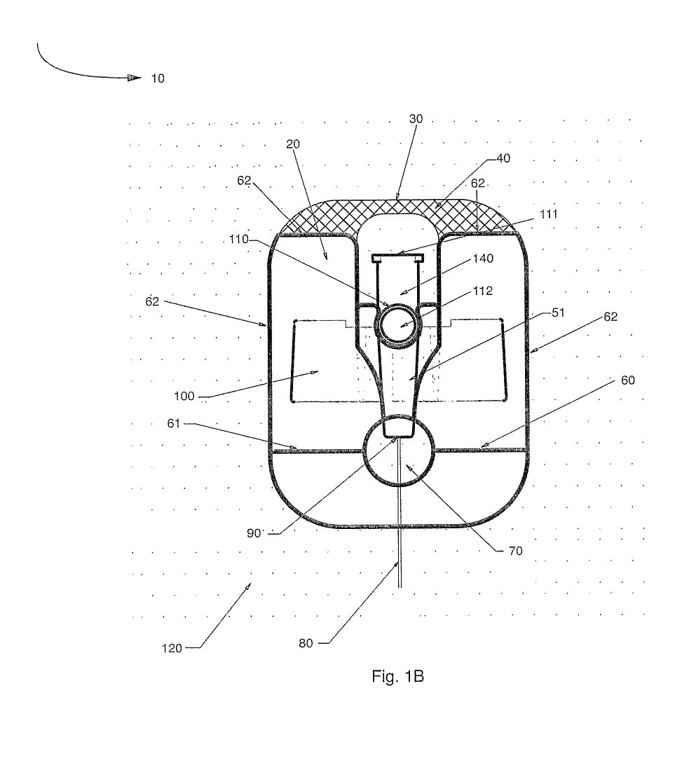

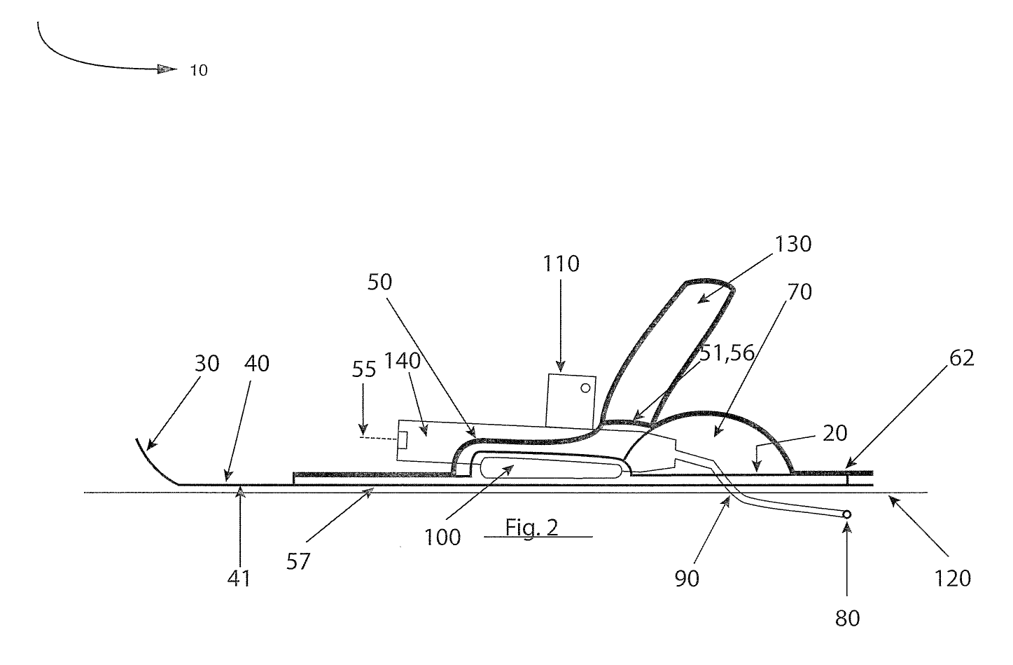

FIG. 1A shows a particular embodiment of the invention (“catheter clip”) for securing a catheter to a skin insertion site of a patient. The catheter clip 10 includes a solid portion 50 joined to a flexible base 20. The flexible base 20 includes an underlying patient contacting surface 41 (see FIG. 2) that is at least partially sealed with a suitable sealing medium such as paper or a plastic. The solid portion 50 includes a clasping means 51 adapted to grasp and securing the catheter to the catheter clip 10. The clasping means 51 may include or consist of nearly any suitable plastic material provided it flexibly resists extension by a user. A cover 40 is in actuating contact with a handle 30 attached to the flexible base 20. The handle 30 and the cover 40 are in sealing contact with a seal formed on at least part of the patient contacting surface 41. Preferably, the seal will protect an adhesive on at least part of the patient contacting surface 41, preferably substantially all of it. The amount of adhesive on the patient contacting surface 41 is not critical provided it is sufficient to attach the catheter clip 10 to the patient.

Referring again to FIG. 1A, the solid portion 50, flexible base 20 and the handle 30 together define an open chamber 52 that includes a distal end 53 (shown contacting a first centerpoint) for receiving the catheter and a proximal end 54 (shown contacting a second centerpoint) for accepting the catheter from the distal end 53 toward the skin insertion site 90 (see FIG. 1B). The first centerpoint of the distal end 53 and the second centerpoint of the proximal end define an axis 55 surrounded by the open chamber 52. In preferred use, a catheter (typically the hard part) is placed along the axis 55 for securement to the catheter clip 10 by the clasping means 51. As shown, the open chamber 52 further includes a top 56 defined by at least part of the solid portion 50 and a bottom 57 (see FIG. 2) opposite the top 56 and defined by the handle 30 and the flexible base 20. In the embodiment shown, the top 56 is in actuating contact with the clasping means 51, wherein actuation of the top 56 is sufficient to flex the clasping means 51 so that it grasps and secures the hard part of a catheter to the catheter clip 10.

The flexible base 20 and optionally the solid portion 50 further define a compartment 70 positioned at or near the insertion site 90. The compartment 70 is typically adapted to at least partially surround the patient insertion site including completely surrounding it. According to FIG. 1A, the top 56 substantially extends from the clasping means 51 to the compartment 70. Optionally, the compartment 70 is in sealing contact with the seal of the patient contacting surface 41. In one embodiment, the compartment 70 will include an effective amount of an antimicrobial (antiseptic) agent sufficient to minimize or eliminate infection at the insertion site. Suitable examples include certain alcohols and phenols known to have antiseptic properties (eg., isopropyl alcohol, phenol), iodine tinctures, chlorhexidine, povidone-iodine (Betadine®) and the like. Preferably, the agents are provided in the compartment 70 in a liquid, semi-liquid or cream format. Topical use antibiotics (eg., bacitracin zinc, polymysin B sulfate, and/or neomycin sulfate), preferably in cream form, can also be used alone or in combination with other antimicrobial agents.

In the embodiment shown in FIG. 1A, the flexible base 20 is substantially planar. Preferably, the flexible base 20 is composed of a material (or composite) of sufficient rigidity so that self-folding is minimized or eliminated. To further minimize self-folding, the catheter clip 10 includes extensions 60, 61, and 62 of the solid portion 50 which are positioned to help keep the flexible base 20 substantially extended. Additional rigidity is provided by firming contact between the extensions 60, 61 and 62 and the flexible base 20.

FIG. 1B shows the catheter clip 10 with a catheter 140 secured to the patient insertion site 90 which catheter 140 is composed of a catheter hard part 110 with a connecting hubs 111, 112, lateral wings 100 for stabilizing the catheter and a catheter soft part 80 extending from the insertion site 90 toward and under the skin 120. The catheter 140 is secured to the catheter clip 10 by actuation of clasping means 51 as previously described. The catheter clip 10 is secured to the patient through the adhesive of the patient contacting surface 41.

Referring now to FIG. 2, the catheter clip 10 is shown in cross-section with the catheter 140 secured to the patient insertion site 90. Also shown is the top 56 of the solid portion 50 with a protrusion 130 that extends acutely from the axis 55 (see also FIG. 1A) and is in actuating contact with the clasping means 51. The protrusion 130 is adapted to receive at least one digit from a user which digit engages protrusion 130 to flex the clasping means 51, grasp the catheter 140 (preferably along the hard part 110), and secure it to the catheter clip 10.

According to one use of the catheter clip 10, a caregiver inserts the soft part 80 of the catheter 140 under the skin 120 at the patient insertion site 90. Subsequently, the caregiver uses a finger to remove cover 40 and the handle 30 in sealing contact with the adhesive along the patient contacting surface 41 of the flexible base 20, thereby removing the seal and exposing the adhesive. The catheter clip 10 is then guided around the patient insertion site 90 to reversibly attach the invention to the skin 120 of the patient. Using the same or different finger, the caregiver engages the protrusion 130 to flex the clasping means 51 and grasp the hard part 110 of the catheter 140, thereby releasably securing the catheter 140 to the catheter clip 10. The procedure is readily reversible. In one approach, the patient contacting surface 41 is removed from the patient. The clamping means 51 is flexed by the user to allow removal of the catheter 140 from the invention.

Manufacture

The invention can be readily made using one or a combination of approaches. In one method, the hard part of the catheter clip 10 is molded as a single-piece system with the clasping means 51 capable of convenient and repeated flexure. The hard part of the catheter clip 10 can be molded from a single polymer material in a one-shot injection molding operation. Many suitable materials could be used such as nearly any moldable plastic that is capable of being formed as provided herein and retaining its shape during use. More specific materials include, but are not limited to, many acrylic and polycarbonate materials, styrenes, and ABS. Other suitable materials include certain polyesters, nylons, and other polymer materials such as certain polyethylenes and polypropylenes. Certain memory plastics may also be used provided intended results are achieved. If two different materials are used, the device can be made by a two-shot process whereby both materials are injected at different gates into the mold cavity. Other processes are also feasible, such as insert molding. MRI compatible materials may be suitable for some invention applications.

Once the hard part of the catheter clip 10 is made, it can be joined using standard approaches to the soft part of the device which generally includes the flexible base 20.

The flexible base 20 can be made using one or a combination of conventional approaches. In one method, the flexible base 20 comprises a relatively thin film, such as a thin urethane or silicone film, adhered to the handle 30 and cover 40. The handle 30 and cover 40 can be made somewhat stiffer than the flexible base 20 facilitate handling. The handle 30 and the cover 40 may extend beyond an edge or edges of the thin film, so that the flexible base 20 can be handled relatively easily. In one embodiment, substantially all of the undersurface of the flexible base 20 (ie., the patient contacting surfacem41) is adhesively coated and contacted with a separate release seal which extends beyond the perimeter of the thin film and is in sealing contact with the handle 30 and cover 40. Nearly any adhesive suitable for topical medical use can be applied to the thin film using standard methods. Examples include, but are not limited to, an acrylic adhesive containing an acrylic acid alkyl ester as a main component, a rubber adhesive containing a natural rubber and/or a synthetic rubber as a main component, as well as “pressure sensitive adhesives” (PSAs). See also U.S. Pat. Nos. 7,094,944; 6,936,661; and 6,805,961 and references disclosed therein for other suitable adhesives.

As will be apparent, the seal may protect the adhesive on at least part of the patient contacting surface 41 (e.g., less than about 50%, for instance, about 5%, 10%, 20% or about 30%), preferably substantially all of the surface (e.g., about 80%, 90%, 95% up to about 100%). The amount of adhesive on the patient contacting surface is not critical provided intended results are achieved.

Additional Embodiments

As will be appreciated, other embodiments of the catheter clip 10 are within the scope of the invention. For instance, and referring now to FIG. 1A, the clasping means 51 is shown as an integral part of the solid portion 50. However in another embodiment, clasping means 51 may be joined to solid portion 50 through an elastic or semi-elastic polymer such as a memory plastic. Alternatively, the clasping means 51 can include or be joined to the solid portion 50 through a spring or related mechanism.

In another embodiment of the catheter clip 10, the top 56 includes means (e.g., a spring or a rod) to position the clasping means 51 to a site desired by the user. It will be appreciated that in other embodiments, the top 56 and the clasping means 51 may be the same component.

In another embodiment, the compartment 70 can be quite small and be just sufficient to surround the insertion site of the catheter. In this embodiment, it will not be necessary to make the compartment 70 so that it is in sealing contact with other components of the device. Also in this embodiment, the use of an antimicrobial may not be necessary. Instead, it may be applied to the skin surface by the user.

Nearly any configuration of the protrusion 130 is acceptable provided intended results are achieved. For instance, and in one embodiment, the protrusion 130 will extend substantially perpendicular to the axis 55 of the open chamber 130.

In yet another embodiment of the catheter clip 10, only one or two of the extensions 60, 61, and 62 will be used to provide rigidity to the flexible base 20. In yet another embodiment, the flexible base 20 will be of sufficient rigidity so that none of extensions 60, 61 and 62 are needed.

### Incorporation by Reference

The contents of all references (including literature references, issued patents, published patent applications, and co-pending patent applications) cited throughout this application are hereby expressly incorporated herein in their entireties by reference.

### Equivalents

It will be appreciated that those skilled in the art, upon consideration of this disclosure, may make modifications and improvements within the spirit and scope of the invention. Moreover, those skilled in the art will recognize, or be able to ascertain using no more than routine experimentation, many equivalents of the specific embodiments of the invention described herein. Such equivalents are intended to be encompassed by the following claims.

1. A device for securing a catheter to an insertion site of a patient comprising:
(a) a flexible base comprising a sealed patient contacting surface;
(b) a solid portion joined to the flexible base and comprising a clasping means for securing the catheter; and
(c) a handle adapted to remove, with one digit, a seal from the patient contacting surface, the handle being joined to the seal, the seal protecting an adhesive on at least part of the surface sufficient to attach the secured catheter to the patient insertion site.

2. The device of claim 1, wherein the solid portion, flexible base and the handle define an open chamber comprising a distal end for receiving the catheter and a proximal end for accepting the catheter from the distal end toward the patient insertion site.

3. The device of claim 2, wherein the distal end of the open chamber comprises a first centerpoint and the proximal end comprises a second centerpoint, the open chamber surrounding an axis defined by the first and second centerpoints.

4. The device of claim 2, wherein the open chamber further comprises: (1) a top defined by the solid portion, and (2) a bottom defined by the handle and the flexible base.

5. The device of claim 1, wherein the flexible base further defines a compartment at or near the patient insertion site, the compartment being adapted to at least partially surround the site.

6. The device of claim 4, wherein the top extends from the clasping means to the compartment.

7. The device of claim 6, wherein at least part of the top comprises a protrusion along an axis substantially perpendicular or acute to the axis of the open chamber.

8. The device of claim 7, wherein the protrusion is adapted to accept at least one digit from a user of the device.

9. The device of claim 8, wherein the top is in actuating contact with the clasping means of the solid portion, wherein actuation of the top is sufficient to secure the clasping means to the catheter.

10. The device of claim 6, wherein the top further comprises means to position the clasping means to a site desired by the user.

11. The device of claim 5, wherein the compartment is in sealing contact with the seal of the patient contacting surface.

12. The device of claim 11, wherein the compartment comprises a therapeutically effective amount of an antimicrobial agent to minimize infection at the insertion site.

13. The device of claim 1, wherein the flexible base is substantially planar and of sufficient rigidity to minimize self-folding.

14. The device of claim 13, wherein the rigidity is provided by firming contact with one or more extensions of the solid portion.

15. A unitary package comprising the device of claim 1, wherein the device is sterile and comprises a packing material substantially resistant to penetration by microorganisms.

16. A method of releasably securing a catheter to an insertion site of a patient comprising:
(a) providing a device comprising:
(i) a flexible base comprising a sealed patient contacting surface,
(ii) a solid portion joined to the flexible base and comprising a clasping means for securing the catheter to the device,
(iii) a handle region joined to the flexible portion and adapted to remove, with one hand, a seal from the patient contacting surface, thereby exposing the adhesive for releasably securing the catheter to the insertion site of the patient;
(b) removing, with at least one digit, the seal from the patient contacting surface of the flexible portion to expose the adhesive;
(c) guiding the device over the catheter;
(d) contacting the flexible base to the patient sufficient to adhere the device thereto; and
(e) actuating the clasping means to grasp the catheter, thereby securing the catheter to insertion site of the patient.

17. (canceled)

18. The device of claim 3, wherein the open chamber further comprises: (1) a top defined by the solid portion, and (2) a bottom defined by the handle and the flexible base.

19. The device of claim 1, wherein the solid portion further defines a compartment at or near the patient insertion site, the compartment being adapted to at least partially surround the site.

20. The device of claim 5, wherein the top extends from the clasping means to the compartment.

21. The device of claim 19, wherein the top extends from the clasping means to the compartment.
